(19) United States
(12) Patent Application Publication
(10) Pub. No.: US 2017/0368044 A1
(43) Pub. Date: Dec. 28, 2017

(54) PIM KINASE INHIBITOR COMBINATIONS

(71) Applicant: Novartis AG, Basel (CH)

(72) Inventors: Zhu Alexander Cao, Acton, MA (US); Abdel Saci, Arlington, MA (US); K. Gary J. Vanasse, Chestnut Hill, MA (US); Joseph Daniel Growney, Reading, MA (US)

(73) Assignee: NOVARTIS AG, Basel (CH)

(21) Appl. No.: 15/698,045

(22) Filed: Sep. 7, 2017

Related U.S. Application Data
(63) Continuation of application No. 14/910,381, filed on Feb. 5, 2016.
(60) Provisional application No. 61/987,664, filed on May 2, 2014.
(60) Provisional application No. 61/912,856, filed on Dec. 6, 2013.
(60) Provisional application No. 61/863,659, filed on Aug. 8, 2013.

(51) Int. Cl. A61K 31/444; A61K 31/4439; A61K 31/519; A61K 45/06

(52) U.S. Cl. CPC A61K 31/444; A61K 45/06; A61K 31/4439; A61K 31/519

(57) ABSTRACT

The present invention relates to a Pim kinase inhibitor compound that can be used alone or in a pharmaceutical combination. One such combination comprises (a) a JAK inhibitor compound, (b) a Pim kinase inhibitor compound, and optionally, at least one pharmaceutically acceptable carrier for simultaneous, separate or sequential use, in particular for the treatment of a myeloid neoplasm or leukemia; a pharmaceutical composition comprising such a combination; the use of such a combination for the preparation of a medicament for the treatment of myeloid neoplasm or leukemia; a commercial package or product comprising such a combination as a combined preparation for simultaneous, separate or sequential use; and to a method of treatment of a mammal, especially a human.

### BACKGROUND OF THE INVENTION

Cancer is the second leading cause of death in the United States. Although “cancer” is used to describe many different types of cancer, e.g., breast, prostate, lung, colon, and pancreatic, each type of cancer differs both at the phenotypic level and the genetic level. The unregulated growth characteristic of cancer occurs when the expression of one or more genes becomes disregulated due to mutations, and cell growth can no longer be controlled.

Myeloproliferative neoplasms (MPNs) are diseases that cause an overproduction of blood cells (platelets, white blood cells and red blood cells) in the bone marrow. MPNs include polycythemia vera (PV), primary or essential thrombocythemia (ET), primary or idiopathic myelofibrosis, chronic myelogenous (myelocytic) leukemia (CML), chronic neutrophilic leukemia (CNL), juvenile myelomonocytic leukemia (JML) and chronic eosinophilic leukemia (CEL)/hyper eosinophilic syndrome (HES). These disorders are grouped together because they share some or all of the following features: involvement of a multipotent hematopoietic progenitor cell, dominance of the transformed clone over the non-transformed hematopoietic progenitor cells, overproduction of one or more hematopoietic lineages in the absence of a definable stimulus, growth factor-independent colony formation in vitro, marrow hypercellularity, megakaryocyte hyperplasia and dysplasia, abnormalities predominantly involving chromosomes 1, 8, 9, 13, and 20, thrombotic and hemorrhagic diatheses, exuberant extramedullary hematopoiesis, and spontaneous transformation to acute leukemia or development of marrow fibrosis but at a low rate, as compared to the rate in CML. The incidence of MPNs varies widely, ranging from approximately 3 per 100,000 individuals older than 60 years annually for CML to 0.13 per 100,000 children from birth to 14 years annually for JML (Vardiman JW et al., Blood 100 (7): 2292-302, 2002). Accordingly, there remains a need for new treatments of MPNs, as well as other cancers such as solid tumors.

### BRIEF SUMMARY OF THE INVENTION

Combinations and uses for N-(4-((1R, 3S,5S)-3-amino-5-methylcyclohexyl)pyridin-3-yl)-6-(2,6-difluorophenyl)-5-fluoropicolinamide, which is shown below as Compound A and disclosed in WO 2010/026124.

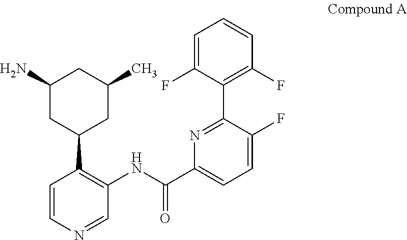 [SMILES: C[C@@H]1C[C@H](N)C[C@H](c2ccncc2NC(=O)c2ccc(F)c(-c3c(F)cccc3F)n2)C1]

In one embodiment of the present invention, there is a pharmaceutical combination comprising a compound that is a JAK inhibitor and a compound that is a Pim inhibitor, more specifically pharmaceutical combination comprising Compound A or a pharmaceutically acceptable salt therefore and ruxolitinib or a pharmaceutically acceptable salt therefore.

Another useful combination of the invention a combination of a Pim inhibitor compound and a PI3K inhibitor compound.

Compound A can also be in combination with an alpha-isoform specific phosphatidylinositol 3-kinase (PI3K) inhibitor shown below as Compound B

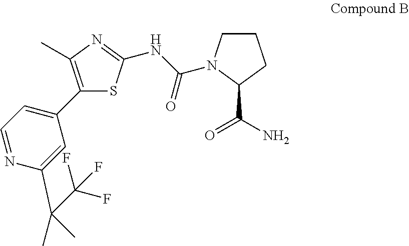 [SMILES: Cc1nc(NC(=O)N2CCC[C@H]2C(N)=O)sc1-c1ccnc(C(C)(C)C(F)(F)F)c1]

Compound B is known by the chemical name (S)-pyrrolidine-1,2-dicarboxylic acid 2-amide 1-({4-methyl-5-[2-(2,2,2-trifluoro-1,1-dimethyl- ethyl)-pyridin-4-yl]-thiazol-2-yl}-amide) or buparlisib. Compound B and its pharmaceutically acceptable salts, their preparation and suitable pharmaceutical formulations containing the same are described in WO 2010/029082, which is hereby incorporated by reference in its entirety. The synthesis of Compound B is described in WO2010/029082 as Example 15.

Other uses for Compound A and combinations are also disclosed.

As shown in WO2010/029082, the Compound B has been found to have significant inhibitory activity for the alpha-isoform of phosphatidylinositol 3-kinases (or PI3K). Compound B has advantageous pharmacological properties as a PI3K inhibitor and shows a high selectivity for the PI3-kinase alpha isoform as compared to the beta and/or delta and/or gamma isoforms.

### BRIEF DESCRIPTION OF THE DRAWINGS

FIG. 1 shows a luminescent cell viability assay of the single agents and combination of ruxolitinib and Compound A in a cell line engineered model of MPN (BA/F3-EpoR-JAK2^{V617F}).

FIG. 2 shows reduction of disease burden in a murine MPN model, BA/F3-EpoR-JAK2^{V617F}, by IVIS Spectrum Preclinical in vivo imaging system (Perkin Elmer).

FIG. 3 shows reductions of spleen size at study endpoint in the murine MPN model BA/F3-EpoR-JAK2^{V617F}.

FIG. 4 shows that Compound A and midostaurin synergize to promote increased apoptosis in AML cell line Molm-13.

FIG. 5 shows that Compound A and midostaurin synergize to inhibit the mTOR pathway in AML cell line Molm-13.

### DETAILED DESCRIPTION OF THE INVENTION

The PIM proteins (Proviral Integration site for the Moloney-murine leukemia virus) are a family of three ser/thr kinases, with no regulatory domains in their sequences and are considered as constitutively active upon their translation (Qian, K. C., et al. J. Biol. Chem. 2004. p 6130-6137). They are oncogenes involved in the regulation of cell cycle, proliferation, apoptosis and drug resistance (Mumenthaler et al. Mol Cancer Ther. 2009; p 2882). Their expression is found particularly elevated in hematopoietic cancers, but some reports have shown an over-expression of PIM1 in pancreatic, prostate and liver cancers as well as a PIM3 expression in certain solid tumors (Reviewed by Alvarado et al, Expert Rev. Hematol. 2012, p 81-96). PIM kinases are regulated by rates of transcription, translation and proteasomal degradation, but the factors that dictate these events are still poorly understood. One pathway that is well established and known to induce PIM1/2 expression is the JAK/STAT signaling pathway (Miura et al, Blood. 1994, p 4135-4141). The STAT proteins are transcription factors, activated downstream of the JAK tyrosine kinases, upon cell surface receptor interaction with their ligands, such as cytokines. Both STAT3 and STAT5 are known to bind to the PIM promoter to induce PIM expression (Stout et al. J Immunol, 2004; 173:6409-6417). Beside the JAK/STATs, the VEGF pathway was also shown to up-regulate PIM expression in endothelial cells during angiogenesis of the ovary, and in human umbilical cord vein cells (Zipo et al, Nat Cell Biol. 2007, p 932-944).

The JAK family plays a role in the cytokine-dependent regulation of proliferation and function of cells involved in immune response. Four mammalian JAK family members are: JAK1 (also known as Janus kinase-1), JAK2 (also known as Janus kinase-2), JAK3 (also known as Janus kinase, leukocyte; JAKL; L-JAK and Janus kinase-3) and TYK2 (also known as protein-tyrosine kinase 2). Aberrant JAK-STAT signaling has been implicated in multiple human pathogenesis. The genetic aberration of JAK2 and the associated activation of STAT in myeloproliferative neoplasia (MPN) is one example of the involvement of this pathway in human neoplasia. Mutation in the upstream thrombopoietin receptor (MPLW525L) and the loss of JAK regulation by LNK (exon 2) have been associated with myelofibrosis (Vainchenker W et al., Blood 2011; 118:1723; Pikman Y et al., Plox Med. 2006, 3: e270). Mutation in JAK2, mostly JAK2 V617F, that leads to constitutive activation of JAK2, have been noted in the majority of patients with primary myelofibrosis (Kralovics R et al., N Engl. J Med 2005, 352: 1779; Baxter E J et al., Lancet 2005, 365: 1054; Levine R L et al., Cancer Cell 2005, 7: 387). Additional mutations in JAK2 axon 12 have been identified in polycythemia vera and idiopathic erythrocytosis (Scott L M et al., N Engl J Med 2007, 356: 459). Additionally, activated JAK-STAT has been suggested as a survival mechanism for human cancers (Hedvat M et al., Cancer Cell 2009; 16: 487). Recently, data have emerged to indicate that JAK2/STATS inhibition would circumvent resistant to PI3K/mTOR blockade in metastatic breast cancer (Britschgi A et al., Cancer Cell 2012; 22: 796). Also, the use of a JAK1/2 inhibitor in IL-6-driven breast, ovarian, and prostate cancers has led to the inhibition of tumor growth in preclinical models (Sansone P and Bromberg J; J. Clinical Oncology 2012, 30: 1005).

Phosphatidylinositol (PI) is a phospholipid that is found in cell membranes. This phospoholipid plays an important role also in intracellular signal transduction. Phosphatidylinositol-3 kinase (PI3K) has been identified as an enzyme that phosphorylates the 3-position of the inositol ring of phosphatidylinositol observations show that deregulation of phosphoinositol-3 kinase and the upstream and downstream components of this signaling pathway is one of the most common deregulations associated with human cancers and proliferative diseases (Parsons et al., Nature 436:792(2005); Hennessey at el., Nature Rev. Drug Dis. 4:988-1004 (2005)). The efficacy of a PI3K inhibitor has been described, for example, in PCT International Patent Application WO 2007/084786.

It has been discovered that administering a JAK inhibitor and a Pim inhibitor combination of the invention provides synergistic effects for treating proliferative diseases of the blood, which can include can myeloid neoplasm, leukemia, other cancers of the blood and could be potentially useful in treating solid cancers as well. Such an approach—combination or co-administration of the two types of agents—can be useful for treating individuals suffering from cancer who do not respond to or are resistant to currently-available therapies. The combination therapy provided herein is also useful for improving the efficacy and/or reducing the side effects of currently-available cancer therapies for individuals who do respond to such therapies.

Certain terms used herein are described below. Compounds of the present invention are described using standard nomenclature. Unless defined otherwise, all technical and scientific terms used herein have the same meaning as is commonly understood by one of skill in the art to which this invention belongs. Compounds of the present invention include their enantiomer forms.

As used herein, the term “pharmaceutically acceptable salts” refers to the nontoxic acid or alkaline earth metal salts of the pyrimidine compounds of the invention. These salts can be prepared in situ during the final isolation and purification of the pyrimidine compounds, or by separately reacting the base or acid functions with a suitable organic or inorganic acid or base, respectively. Representative salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2-hydroxyethanesulfonate, lactate, maleate, methanesulfonate, nicotinate, 2-naphth-alenesulfonate, oxalate, pamoate, pectinate, persulfate, 3-phenylproionate, picrate, pivalate, propionate, succinate, sulfate, tartrate, thiocyanate, p-toluenesulfonate, and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others. Water or oil-soluble or dispersible products are thereby obtained.

Examples of acids that may be employed to form pharmaceutically acceptable acid addition salts include such inorganic acids as hydrochloric acid, hydroboric acid, nitric acid, sulfuric acid and phosphoric acid and such organic acids as formic acid, acetic acid, trifluoroacetic acid, furnaric acid, tartaric acid, oxalic acid, maleic acid, methanesulfonic acid, succinic acid, malic acid, methanesulfonic acid, benzenesulfonic acid, and p-toluenesulfonic acid, citric acid, and acidic amino acids such as aspartic acid and glutamic acid.

Basic addition salts can be prepared in situ during the final isolation and purification of the pyrimidine compounds, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammoniurn, tetraethylamrnonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine, pyridine, picoline, triethanolamine and the like, and basic amino acids such as arginine, lysine and ornithine.

Administration of the combination includes administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agent(s) as compared to the other agent(s) in the combination. Therefore, to permit appropriate dosing, packaged pharmaceutical products may contain one or more dosage forms that contain the combination of agents, and one or more dosage forms that contain one of the combination of agents, but not the other agent(s) of the combination.

The term “single formulation” as used herein refers to a single carrier or vehicle formulated to deliver effective amounts of both therapeutic agents to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

The term “unit dose” is used herein to mean simultaneous administration of both agents together, in one dosage form, to the patient being treated. In some embodiments, the unit dose is a single formulation. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, or patches administered to the patient at the same time. The term “treat” is used herein to mean to relieve, reduce or alleviate, at least one symptom of a disease in a subject. Within the meaning of the present invention, the term “treat” also denotes, to arrest, delay the onset the period prior to clinical manifestation of a disease or symptom of a disease) and/or reduce the risk of developing or worsening a symptom of a disease. The term “subject” is intended to include animals. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from cancer, e.g., myeloproliferative neoplasms or solid tumors.

The term “about” or “approximately” means within 20%, more preferably within 10%, and most preferably still within 5% of a given value or range. Alternatively, especially in biological systems, the term “about” means within about a log (i.e., an order of magnitude) preferably within a factor of two of a given value.

The combination of agents described herein display a synergistic effect. The term “synergistic effect” as used herein, refers to action of two agents producing an effect that is greater than the simple addition of the effects of each drug administered by themselves.

An “effective amount” of a combination of agents is an amount sufficient to provide an observable improvement over the baseline clinically observable signs and symptoms of the depressive disorder treated with the combination.

An “oral dosage form” includes a unit dosage form prescribed or intended for oral administration.

### Methods of Treatment Using Compound A or Combinations of Compound A with a JAK Inhibitor, PI3K Inhibitor or Other Inhibitors

Provided herein are methods of treating cancer, myeloproliferative neoplasms and solid tumors, using Compound A alone or in combination therapy.

Compound A alone or in combination can be used to treat cancer. As used herein, “cancer” refers to any disease that is caused by or results in inappropriately high levels of cell division, inappropriately low levels of apoptosis, or both. Examples of cancer include, without limitation, leukemias (e.g., acute leukemia, acute lymphocytic leukemia, acute myeloid leukemia (AML), also called acute myelocytic leukemia, acute myeloblastic leukemia, acute promyelocytic leukemia, acute myelomonocytic leukemia, acute monocytic leukemia, acute erythroleukemia, chronic leukemia, chronic myelogenous leukemia (CML) also called chronic rnyelocytic leukemia, chronic lymphocytic leukemia (CLL), chronic eosinophilic leukemia, chronic myelomonocytic leukemia, CD19+ leukemia, including CD19+ ALL and CLL), mantle cell leukemia (MCL)), juvenile myelomonocytic leukemia, hypereosinophilic syndrome, systemic mastocytosis, aggressive systemic mastocytosis (ASM), atypical chronic myelogenous leukemia, polycythemia vera, lymphoma (Hodgkin's disease, non-Hodgkin's disease, also known as Hodgkin's lymphoma or non-Hodgkin's lymphoma (NHL), including diffuse large B-cell lymphoma (DLBCL) the most common for of NHL or follicular lymphoma (FL)), Waldenstrom's macroglobulinemia, heavy chain disease, and solid tumors. Compound A alone or in combination can be used for the treatment of myelodysplastic syndromes (MDS).

Furthermore, the therapy provided herein relates to treatment of solid or liquid tumors in warm-blooded animals, including humans, comprising an antitumor-effective dose of Compound A alone or in combination therapy.

The use of Compound A can be alone or in combination therapy for the treatment of solid tumors such as sarcomas and carcinomas including fibrosarcoma, myxosarcoma, liposarcoma, chondrosarcoma, osteogenic sarcoma, chordoma, angiosarcoma, endotheliosarcoma, lymphangio sarcoma, lymphangioendotheliosarcoma, synovioma, mesothelioma, Ewing's tumor, leiomyo sarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, crailiopharyngioma, ependymoma, pinealoma, hemangioblastoma, acoustic neuroma, oligodenroglioma, schwamioma. meningioma, melanoma, neuroblastoma, and retinoblastoma.

In a certain embodiment, the cancer that can be treated using Compound A alone or in combination provided herein is a myeloproliferative disorder or myeloid neoplasm. Myeloproliferative disorders (MPDs), now commonly referred to as meyloproliferative neoplasms (MPNs), are in the class of haematological malignancies that are clonal disorders of hematopoietic progenitors. Tefferi, A. and Vardiman, J. W., Classification and diagnosis of myeloproliferative neoplasms: The 2008 World Health Organization criteria and point-of-care diagnostic algorithms, Leukemia. September 2007, 22: 14-22, is hereby incorporated by reference. They are characterized by enhanced proliferation and survival of one or more mature myeloid lineage cell types. This category includes but is not limited to, chronic myeloid leukemia (CML), polycythemia vera (PV), essential thrombocythemia (ET), myelofibrosis (MF), including primary myelofibrosis (PMF) or idiopathic myelofibrosis, chronic neutrophilic leukemia, chronic eosinophilic leukemia, chronic myelomonocytic leukemia, juvenile myelomonocytic leukemia, hypereosinophilic syndrome, systemic mastocytosis, and atypical chronic myelogenous leukemia. Tefferi, A. and Gilliland, D. G., Oncogenes in myeloproliferative disorders, Cell Cycle, March 2007, 6(5): 550-566 is hereby fully incorporated by reference in its entirety for all purposes.

Compound A of the present invention either alone or in combination can be used to treat refractory or relapsing forms of disease such as relapsed, refractory AML, relapsed, refractory multiple myeloma as well as MDS patients, including in high risk MDS patients.

### Dosages

The optimal dose of Compound A or a combination with Compound A can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages may be established using routine testing and procedures that are well known in the art. Compound A can be dosed alone or in combination at 25 mg, 50 mg, 70 mg, 75 mg, 100 mg, 150 mg, 200 mg, 250 mg, 300 mg, 350 mg 400 mg, 450 mg or 500 mg

In one combination of the invention ruxolitinib can be dosed at 5 mg, 10 mg, 15 mg, 20 mg 25 mg in combination with Compound A being dosed at 25 mg, 50 mg, 70 mg, 75 mg, 100 mg, 150 mg, 200 mg, 250 mg, 300 mg, 350 mg 400 mg, 450 mg or 500 mg. For dosing ranges of in combination of Compound A, ruxolitinib can be 0.25 mg to 25 mg, more preferably 1 mg to 25 mg and Compound A 5 mg to 800 mg, more preferably 20 mg to 200 mg. Once daily dosing is preferred.

In the combination of Compound A and Compound B, for example Compound A can be given in in a standard dose of 200 mg, 300 mg, 400 mg or 500 mg and Compound B in a dose of 100 mg, 200 mg or 300 mg. Optionally depending on patient results Compound A can be given at a lower dose of 100 mg or 70 mg. Because of the pre-clinical synergy shown by the combination of Compound A and Compound B lower clinical doses of each compound may be administered in comparison to the clinical dose of each combination administered together. PKC412 can be dosed at for example between 25-250 mg, with 100 mg being a specific example of this range.

The amount of combination of agents that may be combined with the carrier materials to produce a single dosage form will vary depending upon the individual treated and the particular mode of administration. In some embodiments the unit dosage forms containing the combination of agents as described herein will contain the amounts of each agent of the combination that are typically administered when the agents are administered alone.

Frequency of dosage may vary depending on the compound used and the particular condition to be treated or prevented. In general. the use of the minimum dosage that is sufficient to provide effective therapy is preferred. Patients may generally be monitored for therapeutic effectiveness using assays suitable for the condition being treated or prevented, which will be familiar to those of ordinary skill in the art.

The dosage form can be prepared by various conventional mixing, comminution and fabrication techniques readily apparent to those skilled in the chemistry of drug formulations.

The oral dosage form containing the combination of agents or individual agents of the combination of agents may be in the form of micro-tablets enclosed inside a capsule, e.g. a gelatin capsule. For this, a gelatin capsule as is employed in pharmaceutical formulations can be used, such as the hard gelatin capsule known as CAPSUGEL, available from Pfizer.

Many of the oral dosage forms useful herein contain the combination of agents or individual agents of the combination of agents in the form of particles. Such particles may be compressed into a tablet, present in a core element of a coated dosage form, such as a taste-masked dosage form, a press coated dosage form, or an enteric coated dosage form, or may be contained in a capsule, osmotic pump dosage form, or other dosage form.

The drugs of the present combinations, dosage forms, pharmaceutical compositions and pharmaceutical formulations disclosed herein in a ratio in the range of 100:1 to 1:100.

The optimum ratios, individual and combined dosages, and concentrations of the drug compounds that yield efficacy without toxicity are based on the kinetics of the active ingredients' availability to target sites, and are determined using methods known to those of skill in the art.

The administration of a pharmaceutical combination of the invention may result not only in a beneficial effect, e.g. a synergistic therapeutic effect, e.g. with regard to alleviating, delaying progression of or inhibiting the symptoms, but also in further surprising beneficial effects, e.g. fewer side-effects, an improved quality of life or a decreased morbidity, compared with a monotherapy applying only one of the pharmaceutically active ingredients used in the combination of the invention.

A further benefit may be that lower doses of the active ingredients of the combination of the invention may be used, for example, that the dosages need not only often be smaller but may also be applied less frequently, which may diminish the incidence or severity of side-effects. This is in accordance with the desires and requirements of the patients to be treated.

It is one objective of this invention to provide a pharmaceutical composition comprising a quantity, which may be jointly therapeutically effective at targeting or preventing cancer, e.g., a myeloproliferative disorder. In this composition, a compound of formula I and a compound of formula II may be administered together, one after the other or separately in one combined unit dosage form or in two separate unit dosage forms. The unit dosage form may also be a fixed combination.

The pharmaceutical compositions for separate administration of both compounds, or for the administration in a fixed combination, i.e. a single galenical composition comprising both compounds according to the invention may be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g. as indicated above, or in combination with one or more pharmaceutically acceptable carriers or diluents, especially suitable for enteral or parenteral application.

The pharmaceutical compositions or combinations provided herein (i.e., Compound A with a JAK inhibitor such as ruxolitinib or a PI3K inhibitor, such as Compound B) can be tested in clinical studies. Suitable clinical studies may be, for example, open label, dose escalation studies in patients with proliferative diseases. Such studies prove in particular the synergism of the active ingredients of the combination of the invention. The beneficial effects on proliferative diseases may be determined directly through the results of these studies which are known as such to a person skilled in the art. Such studies may be, in particular, be suitable to compare the effects of a monotherapy using the active ingredients and a combination of the invention. In one embodiment, the dose of a Compound A is escalated until the Maximum Tolerated Dosage is reached, and the other compound (e.g. ruxolitinib or Compound B) is administered with a fixed (non-changing) dose. Alternatively, the other compound of in combination with Compound A may be administered in a non-changing dose and the dose of the compound of Compound A may be escalated. Each patient may receive doses of the compounds either daily or intermittently. The efficacy of the treatment may be determined in such studies, e.g., after 12, 18 or 24 weeks by evaluation of symptom scores every 6 weeks.

### Other Combinations and Indications

Compound A can be used to treat other cancers or the indications disclosed herein such as multiple myeloma and relapsed refractory multiple myeloma in combination with other drugs or treatments, including one or more of a targeted therapy drug, lenalidomide, thalidomide, pomalidomide, a protease inhibitor, bortezomib, carfilzomib, a corticosteroid, dexamethasone, prednisone, daratumumab, a chemotherapy drug, an anthracycline, doxorubicin, liposomal doxorubicin, melphalan, bisphosphonate, cyclophosphamide, etoposide, cisplation, carmustine, stem cell transplantation (bone marrow transplantation) and radiation therapy.

Compound A can be used to treat other cancers or the indications disclosed herein such as acute myeloid leukemia (AML) and relapsed refractory AML in combination with other drugs or treatments, including one or more of a targeted therapy drug, midostaurin (PKC 412), lenalidomide, thalidomide, pomalidomide, sorafenib, tipifarnib, quizartinib, decitabine, CEP-701 (Caphalon), SU5416, SU11248, MLN518, L000021648 (Merck) a chemotherapy drug, decitabine, azacytidine, clofarabine, anthracycline, doxorubicin, liposomal doxorubicin, daunorubicin, idarubicin, cyatarbine, all-trans retonic acid (ATRA), arsenic trioxide, stem cell transplantation (bone marrow transplantation) and radiation therapy. Mutations in the FMS-like tyrosine kinase 3 (FLT3) gene, which encodes a receptor tyrosine kinase, occur in about 25% of cases of AML, and are being targeted with drugs like midostaurin, sorafenib and quirzartinib, all of which are potential combination partners for Compound A. Other mutated with AML include patients with RAS, targeted with GSK1120212 and MSC193636B and JAK2 targeted with rutuxonib.

### Formulations

The drug combinations provided herein may be formulated by a variety of methods apparent to those of skill in the art of pharmaceutical formulation. The various release properties described above may be achieved in a variety of different ways. Suitable formulations include, for example, tablets, capsules, press coat formulations, and other easily administered formulations.

Suitable pharmaceutical formulations may contain, for example, from about 0.1% to about 99.9%, preferably from about 1% to about 60%, of the active ingredient(s). Pharmaceutical formulations for the combination therapy for enteral or parenteral administration are, for example, those in unit dosage forms, such as sugar-coated tablets, tablets, capsules or suppositories, or ampoules, If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount may be reached by administration of a plurality of dosage units.

In particular, a therapeutically effective amount of each of the combination partner of the combination of the invention may be administered simultaneously or sequentially and in any order, and the components may be administered separately or as a fixed combination. For example, the method of treating a disease according to the invention may comprise (i) administration of the first agent (a) in free or pharmaceutically acceptable salt form and (ii) administration of an agent (b) in free or pharmaceutically acceptable salt form, simultaneously or sequentially in any order, in jointly therapeutically effective amounts, preferably in synergistically effective amounts, e.g., in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition.

### Example 1

### In Vitro Assay

Ba/F3-JAK2^{V617F} were grown in DMEM with 10% FBS. Cell viability was determined by measuring cellular ATP content using the CELLTITER-GLO® Luminescent Cell Viability Assay (Promega #G7573) (“the assay”) according to manufacturer's protocol. The assay quantitatively determines the amount of ATP present in a well plate, which is an indicator of metabolically active cells.

Cells were plated on 96-well plates, in triplicates and in growth media. Cells were then treated with ruxolitinib, Compound A or a combination of Cmpd A and ruxolitinib in a ten point dose titration curve (2.7 uM top concentration and 0.45 nM bottom concentration for Ba/F3-JAK2^{V617F}) and incubated at 37 degrees. After 72 hours of incubation, the CellTiter-Glo was added to lyse the cells and measure the ATP consumption. The signal was measured using luminescence intensity recorded on an Envision plate reader.

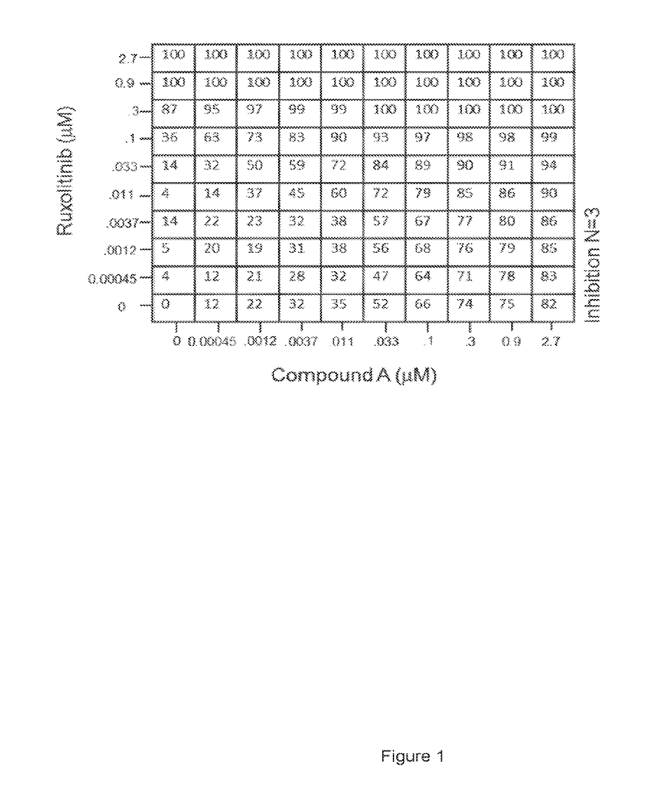

Significant synergy between Cmpd A and ruxolitinib is shown in Ba/F3-JAK2^{V617F} by FIG. 1. The combination of ruxolitinib and Compound A induced greater inhibition of cellular growth, even at very low doses, than either single agents alone. The combination of ruxolitinib (at 0.033 microM) and Compound A (at 0.033 microM) resulted in cell growth inhibition 84%, which essentially equivalent to what was achieved by ruxolitinib alone at 0.3 microM (87%) or Compound A alone at 2.7 microM (84%). This demonstrates a synergistic effect that is almost an order of magnitude improvement over ruxolitinib alone and more than an order of magnitude over Compound A alone.

The MPN cell lines SET2, UKE-1 and AML cell lines HEL92 and CMK also showed similar synergistic effects with this combination. Indeed, combining very low concentrations of compound A and ruxolitinib (33 to 100 nanomolar range) could induce as much inhibition as the use of single agent ruxolitinib alone at higher doses (close to 0.3 to 1 microM range). Accordingly, molecular mechanism analysis has shown that the two compounds synergized in inhibiting various targets in vitro, including the phosphorylation of ribosomal S6 protein, 4eBP1, Bad, ERK1/2, MCL1 expression/degradation and PARP cleavage.

### Example 2

### In Vivo Models

The combination of ruxolitinib and Compound A was further examined in a mouse model of MPN. In this model Ba/F3 cells harbored Epo Receptor and JAK2 V617F mutations. Ba/F3-EpoR-JAK2^{V617F} was engineered with a luciferase tag for experimental imaging. Female SCID/Beige mice were inoculated with 1×10e6 Ba/F3-EpoR-JAK2^{V617F} cells through the tail vein. Systemic disease burden was monitored with IVIS xenogen technology. Disease burden is defined as the sum of dorsal and ventral photon signal. On day 3, disease-bearing mice were randomized into treatment cohort, based on the disease burden. Mice were treated with vehicle, Compound A at 25 mg/kg, by oral gavage (PO) daily (CSD), ruxolitinib at 60 mg/kg, PO, twice daily (BID) or the combination of both agents. The study reached endpoint on after 10 days of treatment. Spleen weight from each of the study cohorts was obtained at endpoint. Relative spleen weight was calculated by normalizing individual spleen weight against the mean spleen weight of the cohort receiving vehicle treatment. The combination of ruxolitinib and Compound A resulted in more pronounced reduction in disease burden and spleen weight than would be expected only from an additive effect of the two compounds.

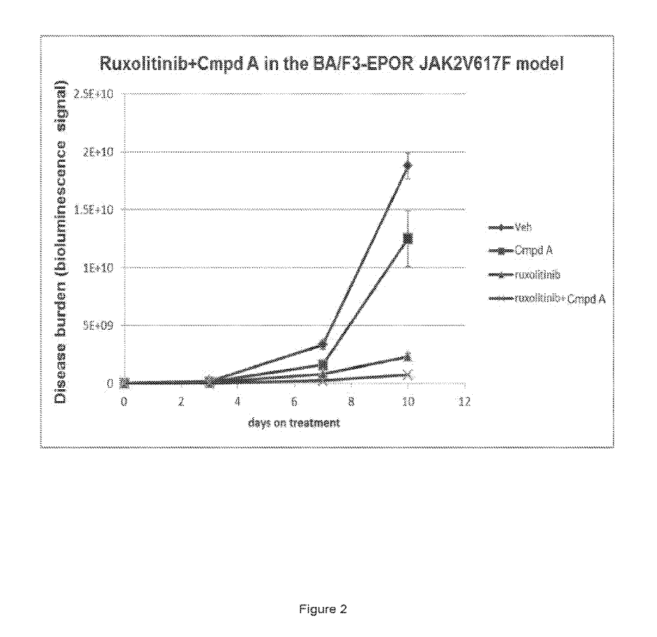

In FIG. 2, the disease burden, measured by the level of bioluminescene, was reduced with ruxolitinib treatment. It was further reduced by ˜3 fold with the combination of ruxolitinib and Compound A.

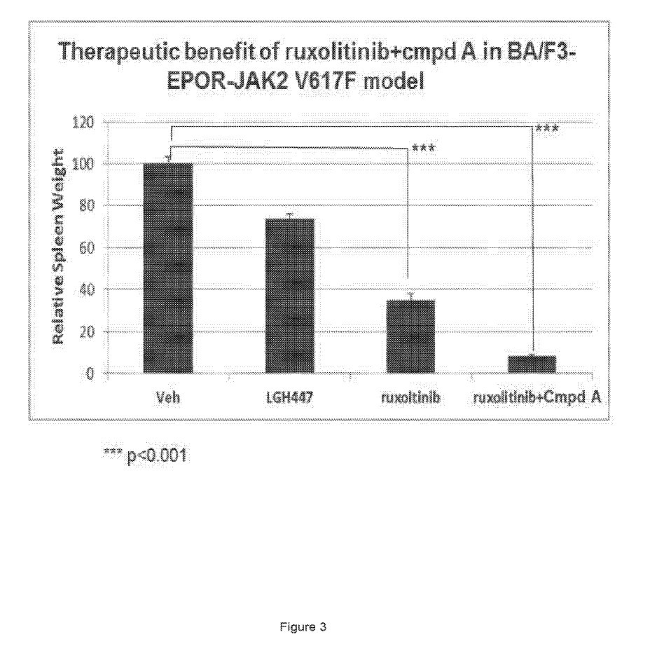

FIG. 3 shows the effects of ruxoltinib and the combination of ruxolitinib with Compound A on spleen size (weight) in the MPN preclinical model. Ruxolitinib monotherapy resulted in ˜65% reduction of spleen weight, relative to that of the vehicle control. The combination of ruxolitinib and Compound A lead to another 4 fold reduction in spleen weight, resulting in relative spleen weight of 8%, relative to that of the vehicle control.

### Example 3

Compound A has shown surprising PK exposure (Cₘₐₓ AUC) properties for its dosage. At 500 mg Compound A was absorbed with peak drug concentrations at range of 3-8 hrs post dose on Day 1, with PK exposure (Cₘₐₓ AUC) over proportional at a dose range of 70mg-250 mg. On Day 14 (steady state), PK exposure seems to form a plateau from 200 mg to 350 mg dose. Exposure at 500 mg (steady state) was increased by about 2-fold compared to that observed from 200 mg to 350 mg dose.

### Example 4

Screening of the combination of Compound A and Compound B in an extended panel of 16 multiple myeloma cell lines showed synergy in all cell lines tested. Furthermore, when this combination was compared to a number of other combinations using a subset of six multiple myeloma cell lines, it was found to be the most synergistic combination. The other combinations screened were Compound A with AUY922, CDZ173, INC424, LBH589, LEE011 or TKI258. The cell lines in which these combination were screened KMM-1, MKS-11, KMS-26, KMS-34, MM1-S, and OPM-2. Only the combination of Compound A and Compound B showed syngergistic in all six of these cell lines.

### Example 5

In vivo studies in mouse xenograft models, KMS-12-BM and KMS-34, further support the synergistic nature of Compound A and Compound B in combination therapy. In the KMS-34 model, Compound A 50 mg/kg in combination with Compound B 20 mg/kg or Compound A 75 mg/kg in combination with Compound B 1 mg/kg resulted in greater anti-tumor activity, relative to the dose matched monotherapy. In the KMS-12-BM model, Compound A monotherapy (100, 75 and 50 mg/kg) resulted in significant anti-tumor activity, while single agent Compound B did not demonstrate in anti-tumor activity. The combination of Compound A (75 and 50 mg/kg) and Compound B (20 mg/kg) resulted in greater anti-tumor activity than that achieved with dose-matched monotherapies. The efficacy of the combination is comparable to the efficacy achieved with Compound A monotherapy at 100 mg/kg. The result suggests that the combination may have activity in multiple myeloma not sensitive to single agent PI3K inhibitors. The data from both of these models also suggests that the combination therapy may allow lower doses to be administered, thus decreasing the need for dose reductions or interruptions and, potentially, resulting in improved drug tolerability for patients.

### Example 6

Both Compound A and Compound B will be administered on a 28 day cycle. The dose-escalation will begin with 200 mg q.d. Compound A and 100 mg q.d. Compound B. Dose levels will be explored. Both study drugs will be administered on a 28 day cycle. Patients randomized to Compound A alone will receive oral Compound B q.d. continuously on a 28 day cycle. Dosing will be orally at approximately the same time each day. Table 1 below shows various starting dose levels

TABLE 1

Starting Dose

| | | |
|---|---|---|
| −2 | 100 | 70 |
| −1 | 200 | 70 |
| 1 | 200 | 100 |
| 2 | 300 | 100 |
| 3 | 300 | 200 |
| 4 | 300 | 300 |
| 5 | 500 | 300 |
| 6 | 600 | 300 |
| 7 | 600 | 400 |

| Scenario | Compond A/Compound B (mg | Next |
|---|---|---|
| 1 | 200/100 | 400/100 |
| 2 | 200/100 | 200/100 |
| 3 | 200/100 | 200/70 |
| | 200/100 | |
| 4 | 400/100 | 400/200 |
| | 200/100 | |
| 5 | 400/100 | 400/100 |
| | 200/100 | |
| 6 | 400/100 | 300/100 |
| | 200/100 | |
| 7 | 200/100 | 300/100 |
| | 200/100 | |
| | 400/100 | |
| | 400/200 | |
| 8 | | 400/300 |
| | 200/100 | |
| | 400/100 | |
| | 400/200 | |
| 9 | | 300/200 |
| | 200/100 | |
| | 400/100 | |
| | 400/100 | |
| 10 | | 400/100 |
| | 200/100 | |
| | 400/100 | |
| | 400/100 | |
| 11 | | 300/100 |
| | 200/100 | |
| | 200/100 | |
| | 300/100 | |
| 12 | | 400/100 |
| | 200/100 | |
| | 200/100 | |
| | 300/100 | 300/100 |
| 13 | | |
| | 200/100 | |
| | 200/100 | |
| | 200/100 | |
| 14 | | 200/100 |
| | 200/100 | |
| | 200/100 | |
| | 200/100 | |
| 15 | | 200/100 |
| | 200/100 | |
| | 400/100 | |
| | 400/200 | |
| | 400/300 | |
| 16 | | 600/300 |
| | 200/100 | |
| | 400/100 | |
| | 400/200 | |
| | 400/300 | |
| 17 | | 400/200 |
| | 200/100 | |
| | 400/100 | |
| | 400/200 | |
| | 400/300 | |
| | 600/300 | |
| 18 | | 600/400 |

### Example 7

Cells were plated at a density of 10,000 cells per 80 μl of medium per well in 96-well plates (Costar #3904) and incubated overnight prior to compound addition. Compound stock was freshly prepared in the appropriate culture medium and manually added to the plates by electronic multichannel pipette in three replicates. Cells were treated with compound alone or with a combination of Compound A and NVP-PKC412. The viability of cells was assessed after 72 hours of treatment by quantification of cellular ATP levels via Cell Titer Glo (Promega #G7571) according to the manufacturers protocol. Plates were read on a luminescence plate reader (Victor X4, Perkin Elmer). Data were analyzed by Chalice software (http://chalice.zalicus.com/documentation/analyzer/index.jsp) to calculate growth inhibition, inhibition and HSA excess (Zimmermann et al., Drug Discov. Today 12: 34-42 (2007); Lehar et al., Nat. Biotech 27 (7):659-666 (2009)).

Both single agents of Compound A and NVP-PKC412 are active in Molm-13 and MV-4-11, but importantly combining the two agent's yields more than additive magnitudes of response at lower doses. For example, in Molm-13 cell line 0.011 μM of NVP-PKC412 yields 66% growth inhibition and 0.3 μM Compound A gives 49% growth inhibition, but the combination of the two agents at these doses yields a growth inhibition of 80% (Table 3, top left panel). This dose combination represents a Loewe excess inhibition value for 10, as seen in Table 4, bottom left panel.

Tables 3-6 show the FLT3 inhibitor PKC412 in the furtherest to the left column in concentration values of micro moles (μM) starting at 0.1 and ending at zero, reading top to bottom and Compound A the PIM inhibitor in the bottom row starting at 2.7 μM and ending in zero, reading right to left. Each compound is diluted threefold times and the dashes below represent the threefold dilution between each number.

TABLE 3
Dose Matrix MOLM-13, Inhibiton, N = 3

| | | | | | | | | | | |
|---|---|---|---|---|---|---|---|---|---|---|
| 0.1 | 100 | 100 | 100 | 100 | 100 | 100 | 100 | 100 | 100 | 100 |
| | 92 | 93 | 95 | 95 | 95 | 96 | 96 | 97 | 98 | 99 |
| .011 | 66 | 70 | 73 | 73 | 73 | 75 | 78 | 80 | 84 | 89 |
| | 45 | 54 | 61 | 59 | 62 | 66 | 69 | 72 | 74 | 79 |
| 1.2e−3 | 25 | 40 | 52 | 49 | 51 | 56 | 62 | 62 | 64 | 69 |
| | 14 | 29 | 38 | 45 | 46 | 51 | 53 | 61 | 60 | 66 |
| 1.4e−4 | 3 | 21 | 21 | 28 | 29 | 43 | 42 | 43 | 50 | 63 |
| | 1 | 20 | 23 | 22 | 28 | 31 | 41 | 44 | 50 | 60 |
| 1.5e−5 | −4 | 14 | 18 | 24 | 24 | 32 | 36 | 44 | 49 | 60 |
| 0 | 0 | 16 | 21 | 25 | 29 | 37 | 41 | 49 | 49 | 61 |
| | 0 | 4.1e−4 | — | 3.7e−3 | — | 0.033 | — | 0.3 | — | 2.7 |

TABLE 4
Loewe Excess MOLM-13, Inhibtion Vol = 5.04(.25) Chi 2 = 140

| | | | | | | | | | | |
|---|---|---|---|---|---|---|---|---|---|---|
| 0.1 | 100 | 100 | 100 | 100 | 100 | 100 | 100 | 100 | 100 | 100 |
| | 92 | 93 | 95 | 95 | 95 | 96 | 96 | 97 | 98 | 99 |
| .011 | 66 | 70 | 73 | 73 | 73 | 75 | 78 | 80 | 84 | 89 |
| | 45 | 54 | 61 | 59 | 62 | 66 | 69 | 72 | 74 | 79 |
| 1.2e−3 | 25 | 40 | 52 | 49 | 51 | 56 | 62 | 62 | 64 | 69 |
| | 14 | 29 | 38 | 45 | 46 | 51 | 53 | 61 | 60 | 66 |
| 1.4e−4 | 3 | 21 | 21 | 28 | 29 | 43 | 42 | 43 | 50 | 63 |
| | 1 | 20 | 23 | 22 | 28 | 31 | 41 | 44 | 50 | 60 |
| 1.5e−5 | −4 | 14 | 18 | 24 | 24 | 32 | 36 | 44 | 49 | 60 |
| 0 | 0 | 16 | 21 | 25 | 29 | 37 | 41 | 49 | 49 | 61 |
| | 0 | 4.1e−4 | — | 3.7e−3 | — | 0.033 | — | 0.3 | — | 2.7 |

TABLE 5
Dose Matrix MV-4-11 Inhibiton, N = 3

| | | | | | | | | | | |
|---|---|---|---|---|---|---|---|---|---|---|
| 0.1 | 87 | 89 | 88 | 90 | 89 | 92 | 91 | 93 | 93 | 96 |
| | 67 | 70 | 67 | 69 | 67 | 68 | 73 | 77 | 76 | 83 |
| .011 | 51 | 49 | 57 | 55 | 53 | 55 | 59 | 59 | 59 | 63 |
| | 22 | 32 | 33 | 39 | 42 | 47 | 54 | 60 | 58 | 61 |
| 1.2e−3 | 26 | 27 | 29 | 41 | 33 | 47 | 47 | 45 | 50 | 55 |
| | 12 | 22 | 32 | 30 | 37 | 44 | 46 | 50 | 49 | 55 |
| 1.4e−4 | −10 | −7 | 13 | 10 | 11 | 14 | 28 | 33 | 38 | 48 |
| | −4 | 2 | 7 | 7 | 11 | 18 | 28 | 35 | 44 | 47 |
| 1.5e−5 | −2 | 3 | 9 | 4 | 4 | 13 | 25 | 33 | 41 | 54 |
| 0 | 0 | 8 | 5 | 17 | 18 | 16 | 23 | 35 | 40 | 54 |
| | 0 | 4.1e−4 | — | 3.7e−3 | — | 0.033 | — | 0.3 | — | 2.7 |

TABLE 6
Loewe Excess MV4-11 lnhibtion Vol 4.55(.28) Chi 2 = 72

| | | | | | | | | | | |
|---|---|---|---|---|---|---|---|---|---|---|
| 0.1 | 6 | 7 | 7 | 9 | 7 | 10 | 10 | 12 | 12 | 14 |
| | −4 | −1 | −3 | −2 | −4 | −3 | 2 | 6 | 6 | 12 |
| .011 | −1 | −3 | 5 | 3 | 1 | 3 | 6 | 7 | 7 | 10 |
| | −7 | 2 | 3 | 9 | 11 | 13 | 16 | 17 | 11 | 11 |
| 1.2e−3 | 13 | 14 | 15 | 25 | 14 | 23 | 15 | 6 | 4 | 5 |
| | 7 | 16 | 24 | 20 | 23 | 23 | 16 | 12 | 4 | 5 |
| 1.4e−4 | −12 | −11 | 8 | 2 | −1 | −6 | −1 | −4 | −6 | −2 |
| | −5 | 0 | 3 | 0 | −1 | −1 | −1 | −2 | −1 | −3 |
| 1.5e−5 | −2 | 1 | 6 | −3 | −8 | −6 | −4 | −4 | −4 | 5 |
| 0 | 0 | 6 | 1 | 10 | 6 | −3 | −5 | −2 | −5 | 4 |
| | 0 | 4.1e−4 | — | 3.7e−3 | — | 0.033 | — | 0.3 | — | 2.7 |

### Example 8

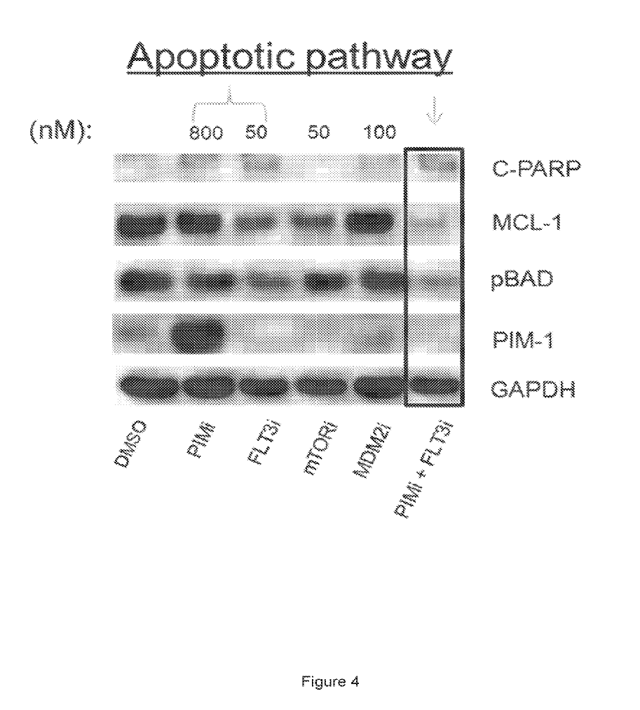

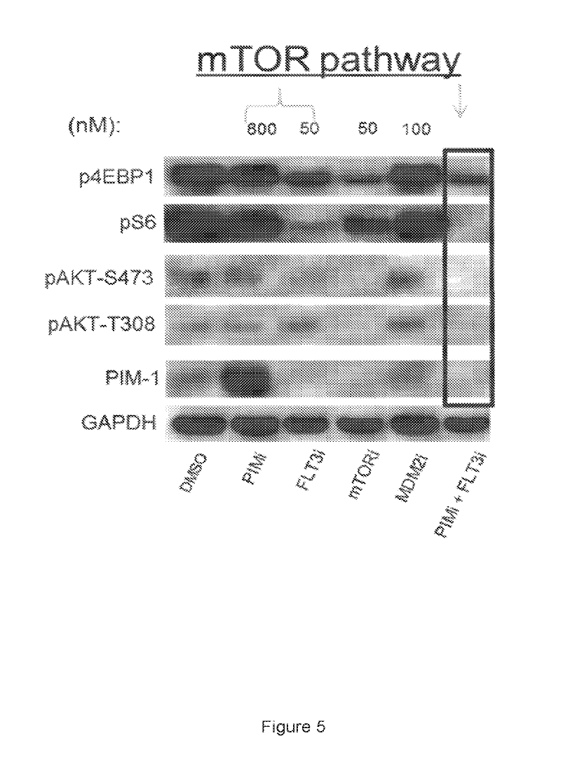

The biochemical profile by protein immunoblot following drug treatment of AML cell line Molm-13 is shown in FIG. 4 and FIG. 5. The AML cells were incubated with 800 nM Compound A (PIM i), 50 nM PKC412 (FLT3i), both compounds combined, or DMSO alone. Cells were lysed after 24 hours of treatment in M-PER mammalian protein extraction buffer containing PhosStop Phosphatase inhibitor cocktail tablet (Roche Diagnostics #04 906 837 001) and Complete Protease Inhibitor cocktail tablet (Roche Diagnostics # 11 836 145 001), Proteins were separated on a 4-12% Bis-Tris NuPAGE SDS gel (Invitrogen #WG1403Bx10) and subsequently transferred to a nitrocellulose membrane using a dry blotting system (Invitrogen iBLOT). Proteins were detected with 1:1000 dilutions of anti-p4EBP1 (Cell Signaling Technologies #9459), anti-pBAD (Cell Signaling Technologies # 9296), anti-Cleaved Parp (Cell Signaling Technologies # 5625), anti-MCL-1 (Cell Signaling Technologies #5453), anti-pAKT-S473 (Cell Signaling Technologies #4058), anti-pAKT-T308 (Cell Signaling Technologies #4056), anti-pS6 (Cell Signaling Technologies #4858), anti-PIM1 (Novartis in-house antibody Batch #NOV22-39-5), and anti-GAPDH (Cell Signaling Technologies #2118). All proteins were detected using an anti-rabbit-HRP secondary antibody and developed with SuperSignal West Dura Chemiluminescent Substrate (Thermo Scientific # 34076) on a Syngene imaging system.

The biochemical effect of compound treatment on apoptotic markers in Molm-13 cell line is demonstrated in FIG. 4. The combination of Compound A (PIMi) plus PKC412 (FLT3i) results in greater degradation of MCL-1 and pBAD, compared to either single agent alone, The biochemical effect on mTOR pathway proteins is demonstrated in FIG. 5. The combination of Compound A plus NVP-PKC412 attenuates p-AKT-S473, pS6 and 4EBP1.

We claim:

1. A pharmaceutical combination comprising ruxolitinib or a pharmaceutically acceptable salt therefore and N-(4-((1R,3S,5S)-3-amino-5-methylcyclohexyl)pyriclin-3-yl)-6-(2,6-difluorophenyl)-5-fluoropicolinamide (Compound A) or a pharmaceutically acceptable salt therefore.

2. The use of the combination of claim 1 for the treatment of a myeloid neoplasm or leukemia.

3. The use of the combination of claim 2, wherein the myeloid neoplasm is a myeloproliferative neoplasm (MPN), a chronic myelogenous leukemia (CML), chronic neutrophilic leukemia, polycythemia vera (PV), myelofibrosis, primary myelofibrosis (PM), idiopathic myleofibrosis, essential thrombocythemia (ET), chronic eosinophilic acute leukemia, mastocytosis, a leukemia, myelodysplastic syndromes (MDS), acute myeloid leukemia (AML), chronic eosinophilic leukemia, chronic myelomonocytic leukemia, juvenile myelomonocytic leukemia, hypereosinophilic syndrome, systemic mastocytosis, and atypical chronic myelogenous leukemia.

4. The use of the combination of claim 3 for the treatment of myeloid neoplasm or leukemia with the concurrent or sequential treatment of ruxolitinib and Compound A.

5. The use of the combination of claim 1 for the treatment of myelodysplastic syndromes (MDS).

6. A method of treating myeloid neoplasm, leukemia or MDS to a patient, comprising administering a compound of claim 1 to the patient.

7. The method of claim 1 wherein the compound is Compound A.

8. The method of claim 7 wherein the leukemia is acute myeloid leukemia (AML).

9. The method of claim 8 wherein the AML is replased or refractory.

10. A combination comprising N-(4-((1R,3S,5S)-3-amino-5-methylcyclohexyl)pyridin-3-yl)-6-(2,6-difluorophenyl)-5-fluoropicolinamide (Compound A) and one or more of a targeted therapy drug, lenalidomide, thalidomide, pomalidomide, a protease inhibitor, bortezomib, carfilzomib, a corticosteroid, dexamethasone, prednisone, daratumumab, a chemotherapy drug, an anthracycline, doxorubicin, liposomal doxorubicin, melphalan, bisphosphonate, cyclophosphamide, etoposide, cisplation, carmustine, stem cell transplantation (bone marrow transplantation), radiation therapy or (S)-pyrrolidine-1,2-dicarboxylic acid 2-amide 1-({4-methyl-5-[2-(2,2,2-trifluoro-1,1-dimethyl-ethyl)- pyridin-4-yl]-thiazol-2-yl}-amide) (Compound B).

11. The combination of claim 10 for the treatment of multiple myeloma.

12. The combination of claim 11 wherein the multiple yeloma is relapsed or refractory.

13. A combination comprising N-(4-((1R,3S,5S)-3-amino-5-methylcyclohexyl)pyridin-3-yl)-6-(2,6-difluorophenyl)-5-fluoropicolinamide (Compound A) and one or more of a targeted therapy drug, midostaurin, lenalidomide, thalidomide, pomalidomide, sorafenib, tipifarnib, quizartinib, decitabine, a chemotherapy drug, decitabine, azacytidine, clofarabine, anthracycline, doxorubicin, liposomal doxorubicin, daunorubicin, idarubicin, cyatarbine, all-trans retonic acid (ATRA), arsenic trioxide, stem cell transplantation (bone marrow transplantation), radiation therapy or (S)-pyrrolidine-1,2-dicarboxylic acid 2-amide 1-({4-methyl-5-[2-(2,2,2-trifluoro-1,1-dimethyl-ethyl)-pyridin-4-yl]-thiazol- 2-yl}-amide).

14. The combination of claim 13 for the treatment of acute myeloid leukemia (AML).

15. The combination of claim 14 wherein the AML is relapsed or refractory.

16. An method of causing a PK exposure plateau to form comprising administered (S)-pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) in a dose of 200 mg to 350 mg.

17. The method of claim 16 wherein the dose is 200 mg, 250 mg, 300 mg, or 350 mg,

18. The combination of claim 11 wherein the dose of Compound A is between 70 to 600 mg once per day and the dose of Compound B is between 100 to 300 mg once per day.
